(19) United States
(12) Patent Application Publication
(10) Pub. No.: US 2003/0109488 A1
(43) Pub. Date: Jun. 12, 2003

(54) Methods for treating dry eye

(75) Inventors: John M. Yanni, Burleson, TX (US); Daniel A. Gamache, Arlington, TX (US)

Correspondence Address: Alcon Research, Ltd., Patrick M. Ryan(Q-148), R&D Counsel, 6201 South Freeway, Fort Worth, TX, 76134-2099, US

(73) Assignee: Alcon, Inc.

(21) Appl. No.: 10/255,205

(22) Filed: Sep. 26, 2002

Related U.S. Application Data
(60) Provisional application No. 60/328,692, filed on Oct. 11, 2001.

(51) Int. Cl. A61K 31/7072; A61K 31/202

(52) U.S. Cl. 514/50; 514/560

(57) ABSTRACT

Methods of treating dry eye by administering fixed combinations of MUC-1 secretagogues, such as HETE derivatives, and lacrimal gland stimulants are disclosed.

This application claims priority to U.S. Provisional Application, Serial No. 60/328,692, filed Oct. 11, 2001.

The present invention is directed to methods for treating dry eye. The methods comprise administering compositions containing combinations of mucin-1 secretagogues and lacrimal gland stimulants.

### BACKGROUND OF THE INVENTION

Dry eye, also known generically as keratoconjunctivitis sicca, is a common ophthalmological disorder affecting millions of Americans each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease and cicatricial pemphigoid manifest dry eye complications.

Although it appears that dry eye may result from a number of unrelated pathogenic causes, all presentations of the complication share a common effect, that is the breakdown of the pre-ocular tear film, which results in dehydration of the exposed outer surface and many of the symptoms outlined above (Lemp, Report of the National Eye Institute/Industry Workshop on Clinical Trials in Dry Eyes, The CLAO Journal, volume 21, number 4, pages 221-231 (1995)).

Practitioners have taken several approaches to the treatment of dry eye. One common approach has been to supplement and stabilize the ocular tear film using so-called artificial tears instilled throughout the day. Other approaches include the use of ocular inserts that provide a tear substitute or stimulation of endogenous tear production.

Examples of the tear substitution approach include the use of buffered, isotonic saline solutions, aqueous solutions containing water soluble polymers that render the solutions more viscous and thus less easily shed by the eye. Tear reconstitution is also attempted by providing one or more components of the tear film such as phospholipids and oils. Phospholipid compositions have been shown to be useful in treating dry eye; see, e.g., McCulley and Shine, Tear film structure and dry eye, Contactologia, volume 20(4), pages 145-49 (1998); and Shine and McCulley, Keratoconjunctivitis sicca associated with meibomian secretion polar lipid abnormality, Archives of Ophthalmology, volume 116(7), pages 849-52 (1998). Examples of phospholipid compositions for the treatment of dry eye are disclosed in U.S. Pat. Nos. 4,131,651 (Shah et al.), 4,370,325 (Packman), 4,409,205 (Shively), 4,744,980 and 4,883,658 (Holly), 4,914,088 (Glonek), 5,075,104 (Gressel et al.), 5,278,151 (Korb et al.), 5,294,607 (Glonek et al.), 5,371,108 (Korb et al.) and 5,578,586 (Glonek et al.). U.S. Pat. No. 5,174,988 (Mautone et al.) discloses phospholipid drug delivery systems involving phospholipids, propellants and an active substance.

Another approach involves the provision of lubricating substances in lieu of artificial tears. For example, U.S. Pat. No. 4,818,537 (Guo) discloses the use of a lubricating, liposome-based composition, and U.S. Pat. No. 5,800,807 (Hu et al.) discloses compositions containing glycerin and propylene glycol for treating dry eye.

Although these approaches have met with some success, problems in the treatment of dry eye nevertheless remain. The use of tear substitutes, while temporarily effective, generally requires repeated application over the course of a patient's waking hours. It is not uncommon for a patient to have to apply artificial tear solution ten to twenty times over the course of the day. Such an undertaking is not only cumbersome and time consuming, but is also potentially very expensive. Transient symptoms of dry eye associated with refractive surgery have been reported to last in some cases from six weeks to six months or more following surgery.

Aside from efforts directed primarily to the alleviation of symptoms associated with dry eye, methods and compositions directed to treatment of the dry eye condition have also been pursued. For example, U.S. Pat. No. 5,041,434 (Lubkin) discloses the use of sex steroids, such as conjugated estrogens, to treat dry eye conditions in post-menopausal women; U.S. Pat. No. 5,290,572 (MacKeen) discloses the use of finely divided calcium ion compositions to stimulate pre-ocular tear film production; and U.S. Pat. No. 4,966,773 (Gressel et al.) discloses the use of microfine particles of one or more retinoids for ocular tissue normalization.

Some recent literature reports suggest that patients suffering from dry eye syndrome disproportionately exhibit the hallmarks of excessive inflammation in relevant ocular tissues, such as the lacrimal and meibomian glands. The use of steroids and cytokine release inhibitors to treat dry eye patients has been disclosed: U.S. Pat. No. 5,958,912; Pflugfelder, et. al. U.S. Pat. No. 6,153,607; and Yanni, J. M.; et. al. WO 0003705 A1. Additionally, cyclosporine A [Tauber, J. Adv. Exp. Med. Biol. 1998, 438 (Lacrimal Gland, Tear Film, and Dry Eye Syndromes 2), 969] has been disclosed for treating dry eye.

Corticosteroids, such as prednisolone and loteprednol, however, cannot be used for prolonged therapy in dry eye patients without causing side effects. Steroid-related complications including increased intraocular pressure and cataract formation have been observed in dry eye patients treated with corticosteroids after several months of therapy. See Marsh, et al., Ophthalmology, 106(4): 811-816 (1999). Marsh, et al. conclude: “Because of the chronic nature of [dry eye] disease and the likelihood of patients developing steroid-related complications with their long-term use, topical nonpreserved methylprednisolone therapy appears to be most appropriate for short-term ‘pulse’ treatment of exacerbations of keratoconjunctivits sicca.” Id. at 811.

Agents claimed for increasing ocular mucin and/or tear production include vasoactive intestinal polypeptide (Dartt et. al., Vasoactive intestinal peptide-stimulated glycocongjugate secretion from conjunctival goblet cells. Experimental Eye Research, volume 63, pages 27-34, (1996)), gefarnate (Nakmura et. al., Gefarnate stimulates secretion of mucin-like glycoproteins by corneal epithelium in vitro and protects corneal epithelium from dessication in vivo, Experimental Eye Research, volume 65, pages 569-574 (1997)), liposomes (U.S. Pat. No. 4,818,537), androgens (U.S. Pat. No. 5,620,921), melanocycte stimulating hormones (U.S. Pat. No. 4,868,154), phosphodiesterase inhibitors (U.S. Pat. No. 4,753,945), and retinoids (U.S. Pat. No. 5,455,265).

U.S. Pat. No. 5,696,166 discloses the use of certain HETE derivatives, including 15-HETE, for treating dry eye and other disorders requiring the wetting of the eye. According to the '166 patent, the HETE derivatives stimulate mucin production and/or secretion in the conjunctival epithelium and goblet cells. Preferably, the HETE derivatives are topically administered to the eye. 15-HETE has been shown to increase the secretion of mucin-1 (MUC-1) from human conjunctival epithelial cells.

### SUMMARY OF THE INVENTION

The present invention is directed to combinations of MUC-1 secretagogues and lacrimal gland stimulants for use in treating dry eye and other disorders requiring the wetting of the eye (disorders that require restoring an intact ocular surface and normal tear function), including symptoms of dry eye associated with refractive surgery such as LASIK surgery. The compositions are preferably administered topically to the eye.

The methods of the present invention provide the advantages of simultaneously treating two aspects of dry eye: stimulating MUC-1 secretion and stimulating the lacrimal glands, which results in protecting the ocular surface epithelium, providing a mucin layer which is essential in holding and spreading of the aqueous and soluble tear components, and enhancing the secretion of these aqueous and soluble tear components. This combination will provide the advantages of requiring less frequent dosing and greater efficacy in a broad range of dry eye patients compared to either MUC-1 secretagogues or lacrimal gland stimulants alone.

### DETAILED DESCRIPTION OF THE INVENTION

The present invention is directed to methods of treating dry eye and other disorders requiring the wetting of the eye by administering compositions comprising a MUC-1 secretagogue and a lacrimal gland stimulant.

As used herein, “MUC-1 secretagogue” means a compound that elicits the production or secretion of MUC-1 by epithelial cells. MUC-1 secretagogues may also elicit production or secretion of other species of mucin, but selectively elicit the production or secretion of MUC-1. Preferred MUC-1 secretagogues are HETE derivatives. “HETE derivative” means a compound selected from the group consisting of the compounds of formulas II-XIV below and pharmaceutically acceptable salts, esters and amides thereof. The most preferred MUC-1 secretagogue is 15(S)-HETE.

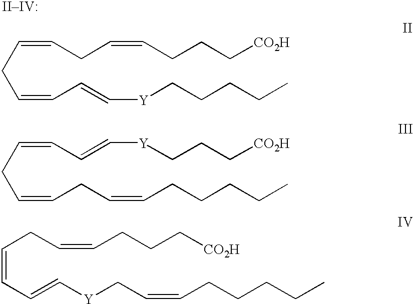

wherein:

Y is C═O (i.e., a carbonyl), or CH(OH) in either configuration, wherein the hydroxy group can be free or functionally modified;

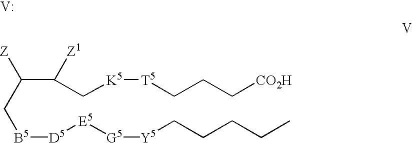

wherein:

Z and Z¹ are H, or ZZ¹ is CH₂;

B⁵-D⁵, E⁵-G⁵ and T⁵-K⁵ are the same or different and are CH₂CH₂, CH═CH, or C≡C;

Y⁵ is C═O (i.e., a carbonyl), or CH(OH) in either configuration, wherein the hydroxy group can be free or functionally modified;

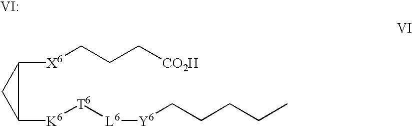

wherein:

X⁶ is CH₂CH₂CH═CH, CH₂CH₂C≡C, CH₂CH₂CH₂CH₂, CH₂CH═CHCH₂, CH₂C≡CCH₂, CH═CHCH₂CH₂, C≡CCH₂CH₂, CH₂CH═C═CH, or CH═C═CHCH₂;

K⁶-T⁶-L⁶ is CH₂CH₂CH₂, CH₂CH═CH, CH₂C≡C, CH═CHCH₂, C≡CCH₂, or CH═C═CH;

Y⁶ is C═O (i.e., a carbonyl), or CH(OH) in either configuration, wherein the hydroxy group can be free or functionally modified;

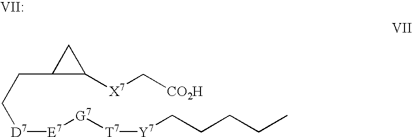

wherein:

X⁷ is CH₂CH₂CH₂, CH₂CH═CH, CH₂C≡C, CH═CHCH₂, C≡CCH₂, or CH═C═CH;

D⁷-E⁷ and G⁷-T⁷ are the same or different and are CH₂CH₂, CH═CH, or C≡C;

Y⁷ is C═O (i.e., a carbonyl), or CH(OH) in either configuration, wherein the hydroxy group can be free or functionally modified;

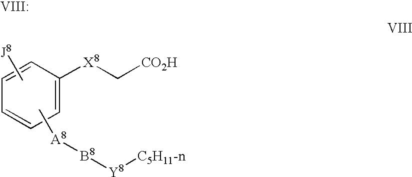

wherein:

X⁸ is C₂-C₅ alkyl, alkynyl, or alkenyl, or a C₃-C₅ allenyl group;

J⁸ is H, free or functionally modified hydroxy group, halo, trihalomethyl, free or functionally modified amino group, free or functionally modified thiol group, C(O)R⁸, or alkyl;

R⁸ is H, OH, alkyl, alkoxy, amino, alkylamino, or alkoxyamino;

A⁸ is direct bond or C₁₋₃ alkyl;

B⁸ is CH₂CH₂, cis- or trans-CH═CH, or C≡C;

Y⁸ is C═O (i.e., a carbonyl), or CH(OH) in either configuration, wherein the hydroxy group can be free or functionally modified;

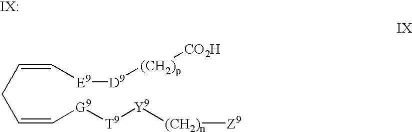

wherein:

E⁹-D⁹ is CH₂CH₂CH₂ or cis-CH₂CH═CH; or E⁹ is trans-CH═CH and D⁹ is CH(OH) in either configuration, wherein the OH is free or functionally modified; or E⁹ is CH₂CH₂ and D⁹ is a direct bond;

p is 1 or 3 when E⁹-D⁹ is CH₂CH₂CH₂ or cis-CH₂CH═CH, or when E⁹ is trans-CH═CH and D⁹ is CH(OH) in either configuration, wherein the OH is free or functionally modified; or p is 0 when E⁹ is CH₂CH₂ and D⁹ is a direct bond;

G⁹-T⁹ is CH₂CH₂, CH(SR)CH₂, or trans-CH═CH;

SR comprises a free or functionally modified thiol group;

n is 0, 2, or 4;

Z⁹ is CH₃, CO₂R⁹, CONR²R³, or CH₂OR⁴;

R⁹ is H or CO₂R⁹ forms a pharmaceutically acceptable salt or a pharmaceutically acceptable ester;

NR²R³ forms a free or functionally modified amino group;

OR⁴ forms a free or functionally modified hydroxy group;

Y⁹ is C═O (i.e., a carbonyl), or CH(OH) in either configuration, wherein the hydroxy group can be free or functionally modified;

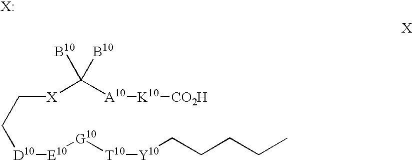

wherein:

K¹⁰ is C₂-C₇ alkyl, alkenyl, or alkynyl, or a C₃-C₇ allenyl group;

A¹⁰ and X¹⁰ are the same or different and are a direct bond, CH₂, NR¹¹, O, or S, with the proviso that at least one of A and X is NR¹¹, O, or S;

B¹⁰ are both H, or B¹⁰B¹⁰ together forms a double bonded O, S, or NR¹², with the proviso that B¹⁰B¹⁰ is a double bonded O, S, or NR¹² when A¹⁰ and X¹⁰ are the same or different and are NR¹¹, O, or S;

NR¹¹ and NR¹² are the same or different and comprise a free or functionally modified amino group;

D¹⁰-E¹⁰ and G¹⁰-T¹⁰ are the same or different and are CH₂CH₂, CH═CH, or C≡C;

Y¹⁰ is C═O (i.e., a carbonyl), or CH(OH) in either configuration, wherein the hydroxy group can be free or functionally modified;

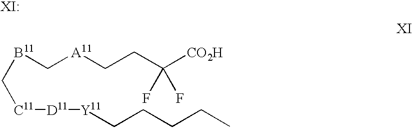

wherein:

A¹¹, B¹¹, C¹¹ and D¹¹ are the same or different and are C₁-C₅ alkyl, alkenyl, or alkynyl, or a C₃-C₅ allenyl group;

Y¹¹ is C═O (i.e., a carbonyl), or CH(OH) in either configuration, wherein the hydroxy group can be free or functionally modified;

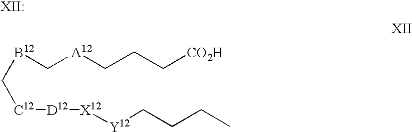

wherein:

A¹², B¹², C¹² and D are the same or different and are C₁-C₅ alkyl, alkenyl, or alkynyl, or a C₃-C₅ allenyl group;

Y¹² is CH(OH) or CCH₃(OH) in either configuration, wherein the hydroxy group can be free or functionally modified, and X¹² is CH₂, CH(CH₃) or C(CH₃)₂; or

Y¹² is CH₂, CH(CH₃) or C(CH₃)₂, and X¹² is CH(OH) or CCH₃(OH) in either configuration, wherein the hydroxy group can be free or functionally modified;

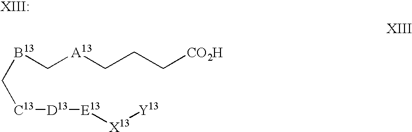

wherein:

A¹³, B¹³, C¹³ and D are the same or different and are C₁-C₅ alkyl, C₂-C₅ alkenyl, C₁-C₅ cyclopropyl, C₂-C₅ alkynyl, or a C₃-C₅ allenyl group;

E¹³ is CH(OH), where the hydroxy group is free or functionally modified;

X¹³ is (CH₂)ₘ or (CH₂)ₘO, wherein m is 1-6, and Y¹³ is a phenyl ring optionally substituted with alkyl, halo, trihalomethyl, acyl, or a free or functionally modified hydroxy, amino, or thiol group; or

X¹³-Y¹³ is (CH₂)ₚY²¹; wherein p is 0-6; and

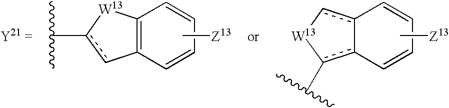

wherein:

W¹³ is CH₂, O, S(O)_{q}, NR¹⁸, CH₂CH₂, CH═CH, CH₂O, CH₂S(O)_{q}, CH═N, or CH₂NR¹⁸; wherein q is 0-2, and R¹⁸ is H, alkyl, or acyl;

Z¹³ is H, alkyl, acyl, halo, trihalomethyl, or a free or functionally modified amino, thiol, or hydroxy group; and

[symbol] is a single or double bond;

or X¹³-Y¹³ is cyclohexyl; and

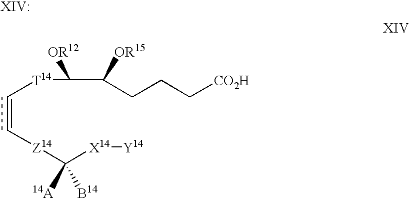

wherein:

OR¹⁴ and OR¹⁵ are the same or different and comprise a free or functionally modified hydroxy group;

G¹⁴, T¹⁴ and Z¹⁴ are the same or different and are CH₂CH₂, cis- or trans-CH═CH or C≡C;

[symbol] is C≡C or cis-CH═CH;

one of A¹⁴, B¹⁴ is H or CH₃, and the other is a free or functionally modified hydroxy group, or A¹⁴-B¹⁴ comprises a double bonded oxygen as a carbonyl, or A¹⁴-B¹⁴ is OCH₂CH₂O;

X¹⁴ is CR¹⁶R¹⁷(CH₂)_{q} or CR¹⁶R¹⁷(CH₂)_{q}O, with q is 0-6;

R¹⁶ and R¹⁷ are the same or different and are H or CH₃;

Y¹⁴ is CH₃, or a phenyl ring optionally substituted with alkyl, halo, trihalomethyl, acyl, or a free or functionally modified hydroxy, thiol, or amino group;

or X¹⁴-Y¹⁴ is (CH₂)ₚY²⁰, p is 0-6,

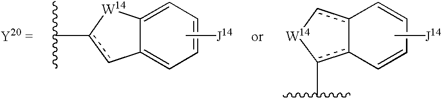

wherein:

W¹⁴ is CH₂, O, S(O)ₘ, NR²¹, CH₂CH₂, CH═CH, CH₂O, CH₂S(O)ₘ, CH═N, or CH₂NR²¹;

m is 0-2;

NR²¹ is NH or a functionally modified amino group;

J¹⁴ is H, alkyl, acyl, halo, trihalomethyl, or a free or functionalized hydroxy, thiol, or amino group; and

[symbol] is a single or double bond;

or X¹⁴-Y¹⁴ is cyclohexyl.

Included within the scope of the present invention are the individual enantiomers of the compounds of formulas II-XIV, as well as their racemic and non-racemic mixtures. The individual enantiomers can be enantioselectively synthesized from the appropriate enantiomerically pure or enriched starting material by means such as those described below. Alternatively, they may be enantioselectively synthesized from racemic/non-racemic or achiral starting materials. (Asymmetric Synthesis; J. D. Morrison and J. W. Scott, Eds.; Academic Press Publishers: New York, 1983-1985, volumes 1-5; Principles of Asymmetric Synthesis; R. E. Gawley and J. Aube, Eds.; Elsevier Publishers: Amsterdam, 1996). They may also be isolated from racemic and non-racemic mixtures by a number of known methods, e.g. by purification of a sample by chiral HPLC (A Practical Guide to Chiral Separations by HPLC; G. Subramanian, Ed.; VCH Publishers: New York, 1994; Chiral Separations by HPLC; A. M. Krstulovic, Ed.; Ellis Horwood Ltd. Publishers, 1989), or by enantioselective hydrolysis of a carboxylic acid ester sample by an enzyme (Ohno, M.; Otsuka, M. Organic Reactions, volume 37, page 1 (1989)). Those skilled in the art will appreciate that racemic and non-racemic mixtures may be obtained by several means, including without limitation, nonenantioselective synthesis, partial resolution, or even mixing samples having different enantiomeric ratios. Also included within the scope of the present invention are the individual isomers substantially free of their respective enantiomers.

As used herein, wavy line attachments indicate that the configuration may be either alpha (α) or beta (β). Hatched lines indicate the α configuration. A solid triangular line indicates the β configuration.

The term “free hydroxy group” means an OH. The term “functionally modified hydroxy group” means an OH which has been functionalized to form: an ether, in which an alkyl, aryl, cycloalkyl, heterocycloalkyl, alkenyl, cycloalkenyl, heterocycloalkenyl, alkynyl, or heteroaryl group is substituted for the hydrogen; an ester, in which an acyl group is substituted for the hydrogen; a carbamate, in which an aminocarbonyl group is substituted for the hydrogen; or a carbonate, in which an aryloxy-, heteroaryloxy-, alkoxy-, cycloalkoxy-, heterocycloalkoxy-, alkenyloxy-, cycloalkenyloxy-, heterocycloalkenyloxy-, or alkynyloxy-carbonyl group is substituted for the hydrogen. Preferred moieties include OH, OCH₂C(O)CH₃, OCH₂C(O)C₂H₅, OCH₃, OCH₂CH₃, OC(O)CH₃, and OC(O)C₂H₅.

The term “free amino group” means an NH₂. The term “functionally modified amino group” means an NH₂ which has been functionalized to form: an aryloxy-, heteroaryloxy-, alkoxy-, cycloalkoxy-, heterocycloalkoxy-, alkenyl-, cycloalkenyl-, heterocycloalkenyl-, alkynyl-, or hydroxy-amino group, wherein the appropriate group is substituted for one of the hydrogens; an aryl-, heteroaryl-, alkyl-, cycloalkyl-, heterocycloalkyl-, alkenyl-, cycloalkenyl-, heterocycloalkenyl-, or alkynyl-amino group, wherein the appropriate group is substituted for one or both of the hydrogens; an amide, in which an acyl group is substituted for one of the hydrogens; a carbamate, in which an aryloxy-, heteroaryloxy-, alkoxy-, cycloalkoxy-, heterocycloalkoxy-, alkenyl-, cycloalkenyl-, heterocycloalkenyl-, or alkynyl-carbonyl group is substituted for one of the hydrogens; or a urea, in which an aminocarbonyl group is substituted for one of the hydrogens. Combinations of these substitution patterns, for example an NH₂ in which one of the hydrogens is replaced by an alkyl group and the other hydrogen is replaced by an alkoxycarbonyl group, also fall under the definition of a functionally modified amino group and are included within the scope of the present invention. Preferred moieties include NH₂, NHCH₃, NHC₂H₅, N(CH₃)₂, NHC(O)CH₃, NHOH, and NH(OCH₃).

The term “free thiol group” means an NH₂. The term “functionally modified thiol group” means an SH which has been functionalized to form: a thioether, where an alkyl, aryl, cycloalkyl, heterocycloalkyl, alkenyl, cycloalkenyl, heterocycloalkenyl, alkynyl, or heteroaryl group is substituted for the hydrogen; or a thioester, in which an acyl group is substituted for the hydrogen. Preferred moieties include SH, SC(O)CH₃, SCH₃, SC₂H₅, SCH₂C(O)C₂H₅, and SCH₂C(O)CH₃.

The term “acyl” represents a group that is linked by a carbon atom that has a double bond to an oxygen atom and a single bond to another carbon atom.

The term “alkyl” includes straight or branched chain aliphatic hydrocarbon groups that are saturated and have 1 to 8 carbon atoms. The alkyl groups may be interrupted by one or more heteroatoms, such as oxygen, nitrogen, or sulfur, and may be substituted with other groups, such as halogen, hydroxyl, aryl, cycloalkyl, aryloxy, or alkoxy. Preferred straight or branched alkyl groups include methyl, ethyl, propyl, isopropyl, butyl and t-butyl.

The term “cycloalkyl” includes straight or branched chain, saturated or unsaturated aliphatic hydrocarbon groups which connect to form one or more rings, which can be fused or isolated. The rings may be substituted with other groups, such as halogen, hydroxyl, aryl, aryloxy, alkoxy, or lower alkyl. Preferred cycloalkyl groups include cyclopropyl, cyclobutyl, cyclopentyl and cyclohexyl.

The term “C₁-C₅ cyclopropyl” means an alkyl chain of 1 to 5 carbon atoms containing a cyclopropyl group wherein the cyclopropyl group may start, be contained in or terminate the alkyl chain.

The term “heterocycloalkyl” refers to cycloalkyl rings that contain at least one heteroatom such as O, S, or N in the ring, and can be fused or isolated. The rings may be substituted with other groups, such as halogen, hydroxyl, aryl, aryloxy, alkoxy, or lower alkyl. Preferred heterocycloalkyl groups include pyrrolidinyl, tetrahydrofuranyl, piperazinyl, and tetrahydropyranyl.

The term “alkenyl” includes straight or branched chain hydrocarbon groups having 1 to 8 carbon atoms with at least one carbon-carbon double bond, the chain being optionally interrupted by one or more heteroatoms. The chain hydrogens may be substituted with other groups, such as halogen. Preferred straight or branched alkenyl groups include, allyl, 1-butenyl, 1-methyl-2-propenyl and 4-pentenyl.

The term “cycloalkenyl” includes straight or branched chain, saturated or unsaturated aliphatic hydrocarbon groups which connect to form one or more non-aromatic rings containing a carbon-carbon double bond, which can be fused or isolated. The rings may be substituted with other groups, such as halogen, hydroxyl, alkoxy, or lower alkyl. Preferred cycloalkenyl groups include cyclopentenyl and cyclohexenyl.

The term “heterocycloalkenyl” refers to cycloalkenyl rings which contain one or more heteroatoms such as O, N, or S in the ring, and can be fused or isolated. The rings may be substituted with other groups, such as halogen, hydroxyl, aryl, aryloxy, alkoxy, or lower alkyl. Preferred heterocycloalkenyl groups include pyrrolidinyl, dihydropyranyl, and dihydrofuranyl.

The term “carbonyl group” represents a carbon atom double bonded to an oxygen atom, wherein the carbon atom has two free valencies.

The term “aminocarbonyl” represents a free or functionally modified amino group bonded from its nitrogen atom to the carbon atom of a carbonyl group, the carbonyl group itself being bonded to another atom through its carbon atom.

The term “lower alkyl” represents alkyl groups containing one to six carbons (C₁-C₆).

The term “halogen” represents fluoro, chloro, bromo, or iodo.

The term “aryl” refers to carbon-based rings which are aromatic. The rings may be isolated, such as phenyl, or fused, such as naphthyl. The ring hydrogens may be substituted with other groups, such as lower alkyl, halogen, free or functionalized hydroxy, trihalomethyl, etc. Preferred aryl groups include phenyl, 3-(trifluoromethyl)phenyl, 3-chlorophenyl, and 4-fluorophenyl.

The term “heteroaryl” refers to aromatic hydrocarbon rings which contain at least one heteroatom such as O, S, or N in the ring. Heteroaryl rings may be isolated, with 5 to 6 ring atoms, or fused, with 8 to 10 atoms. The heteroaryl ring(s) hydrogens or heteroatoms with open valency may be substituted with other groups, such as lower alkyl or halogen. Examples of heteroaryl groups include imidazole, pyridine, indole, quinoline, furan, thiophene, pyrrole, tetrahydroquinoline, dihydrobenzofuran, and dihydrobenzindole.

The terms “aryloxy”, “heteroaryloxy”, “alkoxy”, “cycloalkoxy”, “heterocycloalkoxy”, ”alkenyloxy”, “cycloalkenyloxy”, “heterocycloalkenyloxy”, and “alkynyloxy” represent an aryl, heteroaryl, alkyl, cycloalkyl, heterocycloalkyl, alkenyl, cycloalkenyl, heterocycloalkenyl, or alkynyl group, respectively, attached through an oxygen linkage.

The terms “alkoxycarbonyl”, “aryloxycarbonyl”, “heteroaryloxycarbonyl”, “cycloalkoxycarbonyl”, “heterocycloalkoxycarbonyl”, “alkenyloxycarbonyl”, “cycloalkenyloxycarbonyl”, “heterocycloalkenyloxycarbonyl”, and “alkynyloxycarbonyl” represent an alkoxy, aryloxy, heteroaryloxy, cycloalkoxy, heterocycloalkoxy, alkenyloxy, cycloalkenyloxy, heterocycloalkenyloxy, or alkynyloxy group, respectively, bonded from its oxygen atom to the carbon of a carbonyl group, the carbonyl group itself being bonded to another atom through its carbon atom.

In addition to one or more MUC-1 secretagogues, the compositions administered according to the methods of the present invention comprise one or more lacrimal gland stimulants. As used herein, “lacrimal gland stimulant” means a compound capable of stimulating the release of aqueous tear fluid and soluble tear components from the lacrimal gland. Examples of lacrimal gland stimulants include, but are not limited to, muscarinic agonists, purinergic agonists, and adenosine agonists. Preferred lacrimal gland stimulants are purinergic receptor agonists. Most preferred are the purinergic receptor agonists described in U.S. Pat. No. 5,900,407, especially the compound known as INS-365 (diuridine tetraphosphate, tetrasodium salt).

According to the methods of the present invention, a composition comprising at least one MUC-1 secretagogue, at least one lacrimal gland stimulant and a pharmaceutically acceptable carrier for topical ophthalmic administration or implantation into the conjunctival sac or anterior chamber of the eye is administered to a mammal in need thereof. The compositions are formulated in accordance with methods known in the art for the particular route of administration desired.

Generally, compositions intended to be administered topically to the eye in the form of eye drops or eye ointments will contain approximately 0.00001 to 0.1% of MUC-1 secretagogue and 0.001 to 10% of a lacrimal gland stimulant. Preferably, the MUC-1 secretagogue is a HETE derivative and the amount of HETE derivative is 0.00001 to 0.0001%. The preferred amount of lacrimal gland stimulant is 0.5 to 5%.

The compositions administered according to the present invention may also include various other ingredients, including but not limited to surfactants, tonicity agents, buffers, preservatives, co-solvents and viscosity building agents.

Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 6-7.5.

Compositions formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous compositions which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; and (ii) are safe. Examples or artificial tears compositions useful as artificial tears carriers include, but are not limited to, commercial products, such as Tears Naturale®, Tears Naturale II®, Tears Naturale Free®, and Bion Tears® (Alcon Laboratories, Inc., Fort Worth, Tex.). Examples of phospholipid carrier formulations include those disclosed in U.S. Pat. Nos. 4,804,539 (Guo et al.), 4,883,658 (Holly), 4,914,088 (Glonek), 5,075,104 (Gressel et al.), 5,278,151 (Korb et al.), 5,294,607 (Glonek et al.), 5,371,108 (Korb et al.), 5,578,586 (Glonek et al.); the foregoing patents are incorporated herein by reference to the extent they disclose phospholipid compositions useful as phospholipid carriers of the present invention.

Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P.

Other compounds may also be added to the ophthalmic compositions of the present invention to increase the viscosity or enhance the physical stability of the composition. Examples of viscosity enhancing agents include, but are not limited to: polysaccharides, such as hyaluronic acid and its salts, chondroitin sulfate and its salts, dextrans, various polymers of the cellulose family; carboxy vinyl polymers such as carbomers (e.g., carbomer 974P); and acrylic acid polymers. In general, the phospholipid carrier or artificial tears carrier compositions will exhibit a viscosity of 1 to 400 centipoises (“cps”).

The level of peroxy compounds in HETE derivative raw materials that are used to prepare the pharmaceutical formulations of the present invention may have an impact on the HETE derivative's biological activity. Although the precise relationship has not been defined, it is preferable to use HETE derivative raw material supplies containing peroxy compounds at levels no greater than about 0.3 ppm. Methods for determining peroxy levels are known in the art (e.g., European Pharmacopoeia 1997 3^{rd} Ed., Method 2.5.5—Peroxide Value).

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

The preferred compositions of the present invention are intended for administration to a human patient suffering from dry eye or symptoms of dry eye. Preferably, such compositions will be administered topically. In general, the doses used for the above described purposes will vary, but will be in an effective amount to eliminate or improve dry eye conditions. Generally, 1-2 drops of such compositions will be administered from once to many times per day.

A representative eye drop formulation is provided in Example 1 below.

### EXAMPLE 1

| Ingredient | Amount (% w/v) |
|---|---|
| 15(S)-HETE | 0.00001-0.0001 |
| Lacrimal Gland Stimulant | 0.5-5 |
| Polyoxyl 40 Stearate | 0.1 |
| Boric Acid | 0.25 |
| Carbomer 974P | 0.45 |
| Sodium Chloride | 0.8 |
| Disodium Edetate | 0.01 |
| Benzalkonium Chloride | 0.01 |
| NaOH/HCl | q.s., pH = 7.2 ± 0.2 |
| Purified Water | q.s. 100% |

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

What is claimed is:

1. A composition for the treatment of dry eye and other disorders requiring the wetting of the eye comprising a pharmaceutically acceptable carrier, a pharmaceutically effective amount of a lacrimal gland stimulant and a pharmaceutically effective amount of a MUC-1 secretagogue.

2. The composition of claim 1 wherein the lacrimal gland stimulant is selected from the group consisting of muscarinic agonists; purinergic agonists; and adenosine agonists.

3. The composition of claim 2 wherein the lacrimal gland stimulant is a purinergic receptor agonist.

4. The composition of claim 3 wherein the lacrimal gland stimulant is diuridine tetraphosphate, tetrasodium salt.

5. The composition of claim 1 wherein the MUC-1 secretagogue is a HETE derivative selected from the group consisting of the compounds of formulas II-XIV and pharmaceutically acceptable salts, esters and amides thereof, wherein 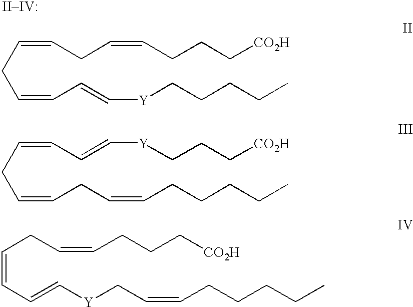
wherein:
Y is C═O (i.e., a carbonyl), or CH(OH) in either configuration, wherein the hydroxy group can be free or functionally modified; 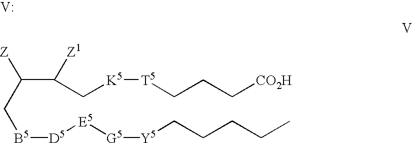
wherein:
Z and Z¹ are H, or ZZ¹ is CH₂;
B⁵-D⁵, E⁵-G⁵ and T⁵-K⁵ are the same or different and are CH₂CH₂, CH═CH, or C≡C;
Y⁵ is C═O (i.e., a carbonyl), or CH(OH) in either configuration, wherein the hydroxy group can be free or functionally modified; 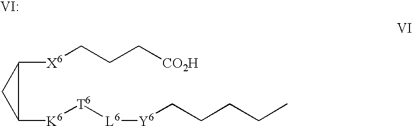
wherein:
X⁶ is CH₂CH₂CH═CH, CH₂CH₂C≡C, CH₂CH₂CH₂CH₂, CH₂CH═CHCH₂, CH₂C≡CCH₂, CH═CHCH₂CH₂, C≡CCH₂CH₂, CH₂CH═C═CH, or CH═C═CHCH₂;
K⁶-T⁶-⁶L is CH₂CH₂CH₂, CH₂CH═CH, CH₂C≡C, CH═CHCH₂, C≡CCH₂, or CH═C═CH;
Y⁶ is C═O (i.e., a carbonyl), or CH(OH) in either configuration, wherein the hydroxy group can be free or functionally modified; 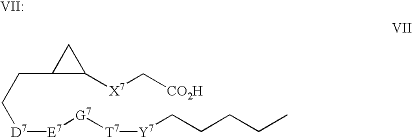
wherein:
X⁷ is CH₂CH₂CH₂, CH₂CH═CH, CH₂C≡C, CH═CHCH₂, C≡CCH₂, or CH═C═CH;
D⁷-E⁷ and G⁷-T⁷ are the same or different and are CH₂CH₂, CH═CH, or C≡C;
Y⁷ is C═O (i.e., a carbonyl), or CH(OH) in either configuration, wherein the hydroxy group can be free or functionally modified; 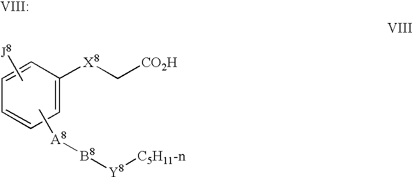
wherein:
X⁸ is C₂-C₅ alkyl, alkynyl, or alkenyl, or a C₃-C₅ allenyl group;
J⁸ is H, free or functionally modified hydroxy group, halo, trihalomethyl, free or functionally modified amino group, free or functionally modified thiol group, C(O)R⁸, or alkyl;
R⁸ is H, OH, alkyl, alkoxy, amino, alkylamino, or alkoxyamino;
A⁸ is direct bond or C₁₋₃ alkyl;
B⁸ is CH₂CH₂, cis- or trans-CH═CH, or C≡C;
Y⁸ is C═O (i.e., a carbonyl), or CH(OH) in either configuration, wherein the hydroxy group can be free or functionally modified; 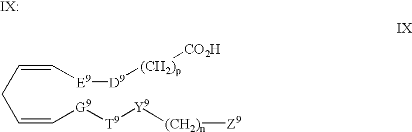
wherein:
E⁹-D⁹ is CH₂CH₂CH₂ or cis-CH₂CH═CH; or E⁹ is trans-CH═CH and D⁹ is CH(OH) in either configuration, wherein the OH is free or functionally modified; or E⁹ is CH₂CH₂ and D⁹ is a direct bond;
p is 1 or 3 when E⁹-D⁹ is CH₂CH₂CH₂ or cis-CH₂CH═CH, or when E⁹ is trans-CH═CH and D⁹ is CH(OH) in either configuration, wherein the OH is free or functionally modified; or p is 0 when E⁹ is CH₂CH₂ and D⁹ is a direct bond;
G⁹-T⁹ is CH₂CH₂, CH(SR)CH₂, or trans-CH═CH;
SR comprises a free or functionally modified thiol group;
n is 0, 2, or 4;
Z⁹ is CH₃, CO₂R⁹, CONR²R³, or CH₂OR⁴;
R⁹ is H or CO₂R⁹ forms a pharmaceutically acceptable salt or a pharmaceutically acceptable ester;
NR²R³ forms a free or functionally modified amino group;
OR⁴ forms a free or functionally modified hydroxy group;
Y⁹ is C═O (i.e., a carbonyl), or CH(OH) in either configuration, wherein the hydroxy group can be free or functionally modified; 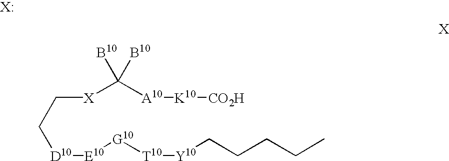
wherein:
K¹⁰ is C₂-C₇ alkyl, alkenyl, or alkynyl, or a C₃-C₇ allenyl group;
A¹⁰ and X¹⁰ are the same or different and are a direct bond, CH₂, NR¹¹, O, or S, with the proviso that at least one of A and X is NR¹¹, O, or S;
B¹⁰ are both H, or B¹⁰B¹⁰ together forms a double bonded O, S, or NR¹², with the proviso that B¹⁰B¹⁰ is a double bonded O, S, or NR¹² when A¹⁰ and X¹⁰ are the same or different and are NR¹¹, O, or S;
NR¹¹ and NR¹² are the same or different and comprise a free or functionally modified amino group;
D¹⁰-E¹⁰ and G¹⁰-T¹⁰ are the same or different and are CH₂CH₂, CH═CH, or C≡C;
Y¹⁰ is C═O (i.e., a carbonyl), or CH(OH) in either configuration, wherein the hydroxy group can be free or functionally modified; 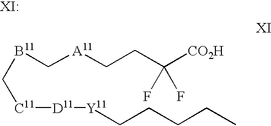
wherein:
A¹¹, B¹¹, C¹¹ and D¹¹ are the same or different and are C₁-C₅ alkyl, alkenyl, or alkynyl, or a C₃-C₅ allenyl group;
Y¹¹ is C═O (i.e., a carbonyl), or CH(OH) in either configuration, wherein the hydroxy group can be free or functionally modified; 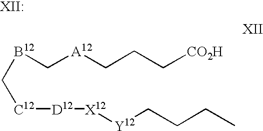
wherein:
A¹², B¹², C¹² and D¹² are the same or different and are C₁-C₅ alkyl, alkenyl, or alkynyl, or a C₃-C₅ allenyl group;
Y¹² is CH(OH) or CCH₃(OH) in either configuration, wherein the hydroxy group can be free or functionally modified, and X¹² is CH₂, CH(CH₃) or C(CH₃)₂; or
Y¹² is CH₂, CH(CH₃) or C(CH₃)₂, and X¹² is CH(OH) or CCH₃(OH) in either configuration, wherein the hydroxy group can be free or functionally modified; 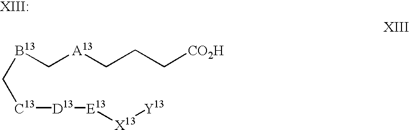
wherein:
A¹³, B¹³, C¹³ and D are the same or different and are C₁-C₅ alkyl, C₂-C₅ alkenyl, C₁-C₅ cyclopropyl, C₂-C₅ alkynyl, or a C₃-C₅ allenyl group;
E¹³ is CH(OH), where the hydroxy group is free or functionally modified;
X¹³ is (CH₂)ₘ or (CH₂)ₘO, wherein m is 1-6, and Y¹³ is a phenyl ring optionally substituted with alkyl, halo, trihalomethyl, acyl, or a free or functionally modified hydroxy, amino, or thiol group; or
X¹³-Y¹³ is (CH₂)ₚY²¹; wherein p is 0-6; and 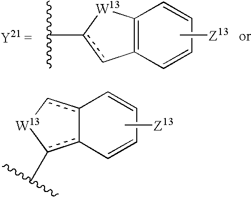
wherein:
W¹³ is CH₂, O, S(O)_{q}, NR¹⁸, CH₂CH₂, CH═CH, CH₂O, CH₂S(O)_{q}, CH═N, or CH₂NR¹⁸; wherein q is 0-2, and R¹⁸ is H, alkyl, or acyl;
Z¹³ is H, alkyl, acyl, halo, trihalomethyl, or a free or functionally modified amino, thiol, or hydroxy group; and [symbol]
is a single or double bond;
or X¹³-Y¹³ is cyclohexyl; and 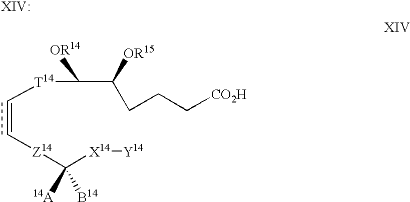
wherein:
OR¹⁴ and OR⁵ are the same or different and comprise a free or functionally modified hydroxy group;
G¹⁴, T¹⁴ and Z¹⁴ are the same or different and are CH₂CH₂, cis- or trans-CH═CH or C≡C; [symbol]
is C≡C or cis-CH═CH;
one of A¹⁴, B¹⁴ is H or CH₃, and the other is a free or functionally modified hydroxy group, or A¹⁴-B¹⁴ comprises a double bonded oxygen as a carbonyl, or A¹⁴-B¹⁴ is OCH₂CH₂O;
X¹⁴ is CR¹⁶R¹⁷(CH₂)_{q} or CR¹⁶R¹⁷ (CH₂)_{q}O, with q is 0-6;
R¹⁶ and R¹⁷ are the same or different and are H or CH₃;
Y¹⁴ is CH₃, or a phenyl ring optionally substituted with alkyl, halo, trihalomethyl, acyl, or a free or functionally modified hydroxy, thiol, or amino group;
or X¹⁴-Y¹⁴ is (CH₂)ₚY²⁰, p is 0-6, 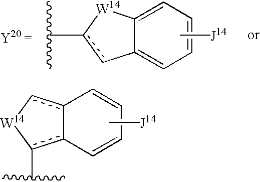
wherein:
W¹⁴ is CH₂, O, S(O)ₘ, NR²¹, CH₂CH₂, CH═CH, CH₂O, CH₂S(O)ₘ, CH═N, or CH₂NR²¹;
m is 0-2;
NR²¹ is NH or a functionally modified amino group;
J¹⁴ is H, alkyl, acyl, halo, trihalomethyl, or a free or functionalized hydroxy, thiol, or amino group; and [symbol]
is a single or double bond; or X¹⁴-Y¹⁴ is cyclohexyl.

6. The composition of claim 2 wherein the pharmaceutically effective amount of lacrimal gland stimulant is 0.001-10%.

7. The composition of claim 5 wherein the pharmaceutically effective amount of MUC-1 secretagogue is 0.00001-0.1%.

8. The composition of claim 1 wherein the MUC-1 secretagogue is 15(S)-HETE and the lacrimal gland stimulant is diuridine tetraphosphate, tetrasodium salt.

9. A method of treating dry eye or other disorders requiring the wetting of the eye comprising topically administering to the eye a composition comprising a pharmaceutically acceptable carrier, a pharmaceutically effective amount of a lacrimal gland stimulant and a pharmaceutically effective amount of a MUC-1 secretagogue.

10. The method of claim 9 wherein the lacrimal gland stimulant is selected from the group consisting of muscarinic agonists; purinergic agonists; and adenosine agonists.

11. The method of claim 10 wherein the lacrimal gland stimulant is a purinergic receptor agonist.

12. The method of claim 11 wherein the lacrimal gland stimulant is diuridine tetraphosphate, tetrasodium salt.

13. The method of claim 9 wherein the MUC-1 secretagogue is a HETE derivative selected from the group consisting of the compounds of formulas II-XIV and pharmaceutically acceptable salts, esters and amides thereof, wherein 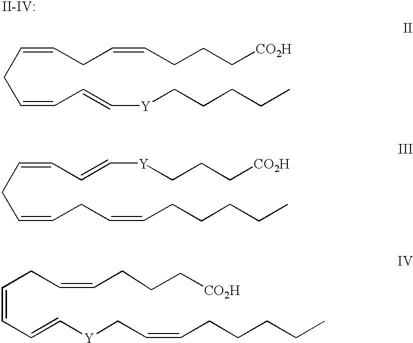
wherein:
Y is C═O (i.e., a carbonyl), or CH(OH) in either configuration, wherein the hydroxy group can be free or functionally modified; 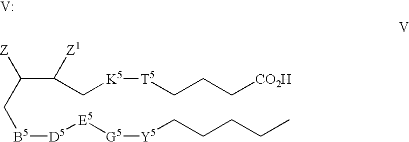
wherein:
Z and Z¹ are H, or ZZ¹ is CH₂;
B⁵-D⁵, E⁵-G⁵ and T⁵-K⁵ are the same or different and are CH₂CH₂, CH═CH, or C≡C;
Y⁵ is C═O (i.e., a carbonyl), or CH(OH) in either configuration, wherein the hydroxy group can be free or functionally modified; 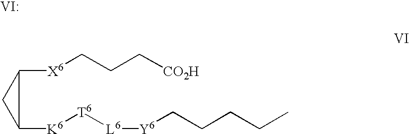
wherein:
X⁶ is CH₂CH₂CH═CH, CH₂CH₂C≡C, CH₂CH₂CH₂CH₂, CH₂CH═CHCH₂, CH₂C≡CCH₂, CH═CHCH₂CH₂, C≡CCH₂CH₂, CH₂CH═C═CH, or CH═C═CHCH₂;
K⁶-T⁶-L⁶ is CH₂CH₂CH₂, CH₂CH═CH, CH₂C≡C, CH═CHCH₂, C≡CCH₂, or CH═C═CH;
Y⁶ is C═O (i.e., a carbonyl), or CH(OH) in either configuration, wherein the hydroxy group can be free or functionally modified; 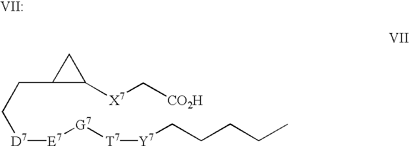
wherein:
X⁷ is CH₂CH₂CH₂, CH₂CH═CH, CH₂C≡C, CH═CHCH₂, C≡CCH₂, or CH═C═CH;
D⁷-E⁷ and G⁷-T⁷ are the same or different and are CH₂CH₂, CH═CH, or C≡C;
Y⁷ is C═O (i.e., a carbonyl), or CH(OH) in either configuration, wherein the hydroxy group can be free or functionally modified; 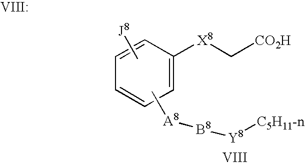
wherein:
X⁸ is C₂-C₅ alkyl, alkynyl, or alkenyl, or a C₃-C₅ allenyl group;
J⁸ is H, free or functionally modified hydroxy group, halo, trihalomethyl, free or functionally modified amino group, free or functionally modified thiol group, C(O)R⁸, or alkyl;
R⁸ is H, OH, alkyl, alkoxy, amino, alkylamino, or alkoxyamino;
A⁸ is direct bond or C₁₋₃ alkyl;
B⁸ is CH₂CH₂, cis- or trans-CH═CH, or C≡C;
Y⁸ is C═O (i.e., a carbonyl), or CH(OH) in either configuration, wherein the hydroxy group can be free or functionally modified; 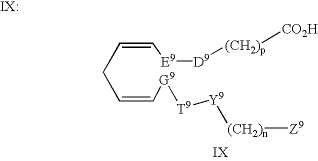
wherein:
E⁹-D⁹ is CH₂CH₂CH₂ or cis-CH₂CH═CH; or E⁹ is trans-CH═CH and D⁹ is CH(OH) in either configuration, wherein the OH is free or functionally modified; or E⁹ is CH₂CH₂ and D⁹ is a direct bond;
p is 1 or 3 when E⁹-D⁹ is CH₂CH₂CH₂ or cis-CH₂CH═CH, or when E⁹ is trans-CH═CH and D⁹ is CH(OH) in either configuration, wherein the OH is free or functionally modified; or p is 0 when E⁹ is CH₂CH₂ and D⁹ is a direct bond;
G⁹-T⁹ is CH₂CH₂, CH(SR)CH₂, or trans-CH═CH;
SR comprises a free or functionally modified thiol group;
n is 0, 2, or 4;
Z⁹ is CH₃, CO₂R⁹, CONR²R³, or CH₂OR⁴;
R⁹ is H or CO₂R⁹ forms a pharmaceutically acceptable salt or a pharmaceutically acceptable ester;
NR²R³ forms a free or functionally modified amino group;
OR⁴ forms a free or functionally modified hydroxy group;
Y⁹ is C═O (i.e., a carbonyl), or CH(OH) in either configuration, wherein the hydroxy group can be free or functionally modified; 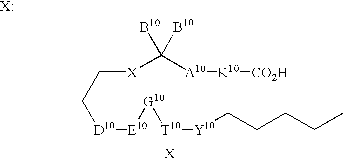
wherein:
K¹⁰ is C₂-C₇ alkyl, alkenyl, or alkynyl, or a C₃-C₇ allenyl group;
A¹⁰ and X¹⁰ are the same or different and are a direct bond, CH₂, NR¹¹, O, or S, with the proviso that at least one of A and X is NR¹¹, O, or S;
B¹⁰ are both H, or B¹⁰B¹⁰ together forms a double bonded O, S, or NR¹², with the proviso that B¹⁰B¹⁰ is a double bonded O, S, or NR¹² when A¹⁰ and X¹⁰ are the same or different and are NR¹¹, O, or S;
NR¹¹ and NR¹² are the same or different and comprise a free or functionally modified amino group;
D¹⁰-E¹⁰ and G¹⁰-T¹⁰ are the same or different and are CH₂CH₂, CH═CH, or C≡C;
Y¹⁰ is C═O (i.e., a carbonyl), or CH(OH) in either configuration, wherein the hydroxy group can be free or functionally modified; 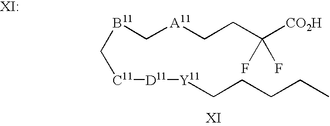
wherein:
A¹¹, B¹¹, C¹¹ and D¹¹ are the same or different and are C₁-C₅ alkyl, alkenyl, or alkynyl, or a C₃-C₅ allenyl group;
Y¹¹ is C═O (i.e., a carbonyl), or CH(OH) in either configuration, wherein the hydroxy group can be free or functionally modified; 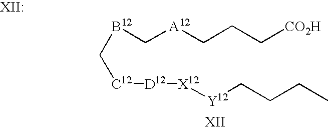
wherein:
A¹², B¹², C¹² and D are the same or different and are C₁-C₅ alkyl, alkenyl, or alkynyl, or a C₃-C₅ allenyl group;
Y¹² is CH(OH) or CCH₃(OH) in either configuration, wherein the hydroxy group can be free or functionally modified, and X¹² is CH₂, CH(CH₃) or C(CH₃)₂; or
Y¹² is CH₂, CH(CH₃) or C(CH₃)₂, and X¹² is CH(OH) or CCH₃(OH) in either configuration, wherein the hydroxy group can be free or functionally modified; 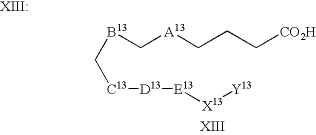
wherein:
A¹³, B¹³, C¹³ and D¹³ are the same or different and are C₁-C₅ alkyl, C₂-C₅ alkenyl, C₁-C₅ cyclopropyl, C₂-C₅ alkynyl, or a C₃-C₅ allenyl group;
E¹³ is CH(OH), where the hydroxy group is free or functionally modified;
X¹³ is (CH₂)ₘ or (CH₂)ₘO, wherein m is 1-6, and Y¹³ is a phenyl ring optionally substituted with alkyl, halo, trihalomethyl, acyl, or a free or functionally modified hydroxy, amino, or thiol group; or
X¹³-Y¹³ is (CH₂)ₚY²¹; wherein p is 0-6; and 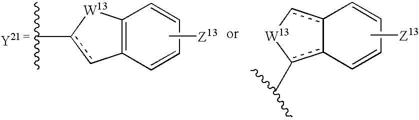
wherein:
W¹³ is CH₂, O, S(O)_{q}, NR¹⁸, CH₂CH₂, CH═CH, CH₂O, CH₂S(O)_{q}, CH═N, or CH₂NR¹⁸; wherein q is 0-2, and R¹⁸ is H, alkyl, or acyl;
Z¹³ is H, alkyl, acyl, halo, trihalomethyl, or a free or functionally modified amino, thiol, or hydroxy group; and [symbol]
is a single or double bond;
or X¹³-Y¹³ is cyclohexyl; and 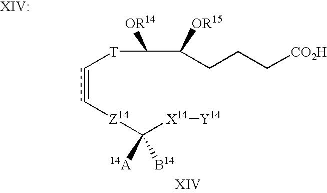
wherein:
OR¹⁴ and OR¹⁵ are the same or different and comprise a free or functionally modified hydroxy group;
G¹⁴, T¹⁴ and Z¹⁴ are the same or different and are CH₂CH₂, cis- or trans-CH═CH or C≡C; [symbol]
is C≡C or cis-CH═CH;
one of A¹⁴, B¹⁴ is H or CH₃, and the other is a free or functionally modified hydroxy group, or A¹⁴-B¹⁴ comprises a double bonded oxygen as a carbonyl, or A¹⁴-B¹⁴ is OCH₂CH₂O;
X¹⁴ is CR¹⁶R¹⁷(CH₂)_{q} or CR¹⁶R¹⁷(CH₂)_{q}O, with q is 0-6;
R¹⁶ and R¹⁷ are the same or different and are H or CH₃;
Y¹⁴ is CH₃, or a phenyl ring optionally substituted with alkyl, halo, trihalomethyl, acyl, or a free or functionally modified hydroxy, thiol, or amino group;
or X¹⁴-Y¹⁴ is (CH₂)ₚY²⁰, p is 0-6, 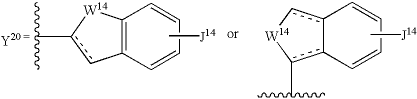
wherein:
W¹⁴ is CH₂, O, S(O)ₘ, NR²¹, CH₂CH₂, CH═CH, CH₂O, CH₂S(O)ₘ, CH═N, or CH₂NR²¹;
m is 0-2;
NR²¹ is NH or a functionally modified amino group;
J¹⁴ is H, alkyl, acyl, halo, trihalomethyl, or a free or functionalized hydroxy, thiol, or amino group; and [symbol]
is a single or double bond;
or X¹⁴-Y¹⁴ is cyclohexyl.

14. The method of claim 10 wherein the pharmaceutically effective amount of lacrimal gland stimulant is 0.001-10%.

15. The method of claim 13 wherein the pharmaceutically effective amount of MUC-1 secretagogue is 0.00001-0.1%.

16. The method of claim 9 wherein the MUC-1 secretagogue is 15(S)-HETE and the lacrimal gland stimulant is diuridine tetraphosphate, tetrasodium salt.
